(19) United States
(12) Patent Application Publication
(10) Pub. No.: US 2007/0021605 A1
(43) Pub. Date: Jan. 25, 2007

(54) PROCESS AND COMPOSITION FOR MAKING OLANZAPINE FORM I

(76) Inventors: Rolf Keltjens, Wijchen (NL); Lambertus Thijs, Wijchen (NL)

Correspondence Address: MARK R. BUSCHER;SYNTHON IP INC, 7130 HERITAGE VILLAGE PLAZA, STE 202, GAINESVILLE, VA, 20155, US

(21) Appl. No.: 11/458,513

(22) Filed: Jul. 19, 2006

Related U.S. Application Data
(60) Provisional application No. 60/700,717, filed on Jul. 20, 2005.

(51) Int. Cl. C07D 498/02

(52) U.S. Cl. 540/560

(57) ABSTRACT

Olanzapine Form I crystals can be made by precipitation from a gas/supercritical fluid composition containing carbon dioxide.

This application claims the benefit of priority under 35 U.S.C. §119(e) from U.S. provisional patent application Ser. No. 60/700,717, filed Jul. 20, 2005, the entire contents of which are incorporated herein by reference.

### BACKGROUND OF THE INVENTION

The present invention relates to a new method of making olanzapine in the crystalline Form I using a fluid containing carbon dioxide.

Olanzapine is represented by the structural formula (1) 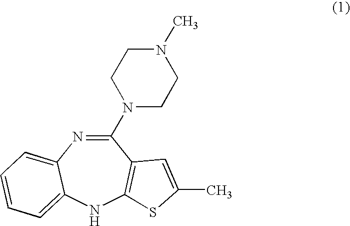
and is a pharmaceutically useful compound. In medical treatments, it is useful as an antipsychotic agent, particularly for the treatment of schizophrenia. The marketed final forms include coated tablets and quick dissolvable tablets. The single tablet comprises from 2.5 to 20 mg of olanzapine.

In the present commercially available final forms the active substance is marketed as a free base. It is a white to yellow crystalline solid that is insoluble in water (solubility at pH 6.8 is about 0.02 mg/ml).

Olanzapine and pharmaceutically acceptable salts have been suggested in EP 454436 and corresponding U.S. Pat. No. 5,229,382. In the final stage of the production process, olanzapine was obtained by a crystallization of the crude olanzapine product from acetonitrile. The patent does not refer to or identify any specific polymorphic crystalline form of olanzapine.

Later, it became known that olanzapine base may exist in various crystalline modifications, including some hydrated/solvated forms, that are stable at ambient conditions (For example, see EP 733635/U.S. Pat. No. 5,736,541, WO 98-11893, and EP 831098).

The term “Form I olanzapine” was later designated in EP 733635 to the anhydrous olanzapine product that was stated to be obtainable according to the process of U.S. Pat. No. 5,229,382.

EP 733635/U.S. Pat. No. 5,736,541 disclose Form II olanzapine which is characterized by a main X-ray powder diffraction peak of d-value 10.26 A. This form has been prepared by crystallizing “technical grade” olanzapine (the product of the earlier synthesis) from ethyl acetate. This form appears to be more stable than the Form I, but it is convertible to Form I. Similarly as Form I, the Form II is an anhydrate.

U.S. Pat. No. 6,348,458 (WO 01/47933) discloses other crystalline polymorphic forms of olanzapine, namely Form III, Form IV and Form V. More recently, WO 03/091260 discloses Form VI olanzapine. US Appl. 2002-0086993 discloses a polymorphic form designated as form X.

As the system used for numbering of known olanzapine forms is sometimes confusing in the prior art disclosures (for instance, the EP 828494 calls as olanzapine Form I a product that is identical with olanzapine Form II of the above definition), the “Form I” of olanzapine as used herein is defined as the solid state form of anhydrous olanzapine base which is characterized by a main peak on the X-ray powder diffraction spectrum of d-value 9.9463 A. The full diffraction pattern of the Form I has been disclosed in EP 733635. The “Form II” of olanzapine as used herein has the same definition as used in EP 733635/U.S. Pat. No. 5,736,541, namely it is characterized by a main X-ray powder diffraction peak of d-value 10.26 A.

Interestingly, WO 02/18390 indicates that upon repetition of the disclosed process in U.S. Pat. No. 5,229,382, the product obtained does not correspond to the Form I. Instead a Form II olanzapine is obtained after the crystallization from acetonitrile, while a hydrated olanzapine is obtained prior to the crystallization. The Form I complying with the above definition was actually prepared in WO 02/18390 by recrystallization of olanzapine Form II or a hydrate of olanzapine from dichloromethane, followed by drying of the wet product at 60-70° C. In fact, the product of crystallization is a dichloromethane solvate of olanzapine, which liberates dichloromethane under the conditions of drying and yields the Form I.

Furthermore, Reutzel-Edens et al. (Crystal Growth and Design, 2003, vol. 3, No. 6,897-907) studied various solid state forms of anhydrous and hydrated forms of olanzapine. They report that while it is possible to prepare pure olanzapine Form II (which is confusingly designated as “Form I” in the article) by a direct crystallization from various solvents, it is impossible to prepare olanzapine Form I (which is designated as “Form II” in the article) in such a way. The Form I is obtainable only by a desolvation of various olanzapine solvates (methanol, dichloromethane and/or chloroform solvates) and such a product is admixed with various other forms of olanzapine. No conditions were identified that would yield pure Form I.

WO 03/97650 purports to prepare essentially pure olanzapine Form I also by a desolvation of various olanzapine solvates. However, based on the published X-ray diffraction pattern, the product appears to not be olanzapine form I as defined herein.

Essentially pure olanzapine Form I was prepared and characterized in WO 03/101997, employing a complicated purification and precipitation process.

WO 04/006933 attempts to prepare olanzapine Form I by a desolvation of various solvates and mixed solvates.

Commonly owned co-pending U.S. patent application Ser. No. 11/050,851, filed Jan. 27, 2005, relates to a process for making olanzapine Form I by heating a solid state olanzapine acetate compound to produce Form I.

The Form I olanzapine is an important product. However, it is desirable to improve the methods of making it. In particular, it is desirable to provide essentially pure olanzapine Form I, free from other polymorphic forms, by a simple and controllable process.

### SUMMARY OF THE INVENTION

The present invention relates to a process for producing crystalline olanzapine of Form I by the use of carbon dioxide as a solvent and to carbon dioxide fluid compositions used in the process. Accordingly, an aspect of the invention relates to a process for making crystalline olanzapine Form I, which comprises reducing the pressure of a gas/supercritical fluid composition comprising carbon dioxide and olanzapine to precipitate crystalline olanzapine form I from said composition. Typically the composition contains supercritical or near supercritical carbon dioxide. The reduction in pressure changes the solubility of olanzapine in the gas and/or supercritical fluid thereby causing precipitation of olanzapine. Surprisingly, such a method can reliably form olanzapine Form I.

### BRIEF DESCRIPTION OF THE DRAWING

FIG. 1 depicts an arrangement of equipment suitable for performing a process of the invention.

### DETAILED DESCRIPTION OF THE INVENTION

The present invention relates to the discovery that olanzapine Form I can be precipitated from carbon dioxide. By reducing the pressure of a gas/supercritical fluid composition comprising carbon dioxide and olanzapine, crystalline olanzapine Form I can be precipitated. The term “gas/supercritical fluid composition” means a single or multi-component composition in a gas and/or a supercritical fluid state. For clarity, a supercritical fluid, as is generally known, refers to a fluid that is at a temperature and pressure equal to or above its critical temperature and critical pressure, respectively. Conceptually a supercritical fluid is a high density gas. The gas/supercritical fluid composition contains carbon dioxide and olanzapine. The carbon dioxide acts as a solvent and olanzapine as the solute dissolved therein. While other ingredients could also be present in the composition, such as an additional solvent, generally the carbon dioxide and olanzapine account for at least 90 mol %, more preferably at least 99 mol % of the fluid composition; i.e. the fluid generally “consists essentially of” carbon dioxide and olanzapine. The gas/supercritical fluid composition is non-liquid by definition. However, this does not rule out the possibility of a liquid being additionally present in carrying out the process of the invention.

To precipitate the olanzapine from the gas/supercritical fluid composition, the pressure of the fluid composition is reduced. Prior to this reduction in pressure, the fluid composition has a relatively high pressure. The use of higher pressure generally increases the solvent power of the fluid; i.e., the solubility of olanzapine in carbon dioxide increases. Typically the pressure is at least 60 bars, more typically at least 74 bars (critical pressure for carbon dioxide), and usually 100 bars to 400 bars or more. In some embodiments, the pressure is within the range of 200 bars to 400 bars, more preferably 250-350 bars. The pressure may be constrained by the equipment involved, the cost/benefit, and to some extent the temperature. Below the critical temperature, the gas can be condensed to form a liquid media if sufficient pressure is applied. This is generally undesirable because a liquid is relatively incompressible and not suited for facilitating pressure dependent solubility changes. A gas that has reached or exceeded its critical temperature cannot be condensed into a liquid, regardless of how much pressure is applied. The critical temperature for carbon dioxide is about 31° C. Thus, at 31° C. or above carbon dioxide can be highly compressed, even approaching/equaling the density of liquid carbon dioxide, without condensing to a liquid; e.g. a high density gas or more precisely a supercritical fluid. At temperatures less than 31° C., the pressure of the carbon dioxide-based fluid composition is limited so as not to completely liquefy the fluid. For example, at room temperature gaseous carbon dioxide will condense to liquid at about 60 bars.

Higher temperatures also generally improve the solvent power of the fluid, thereby increasing the amount of olanzapine that can be dissolved into the fluid composition. However, the temperature can be constrained by the equipment, the cost/benefit, and the thermodynamics in producing olanzapine Form I. Specifically regarding Form I formation, sufficiently high temperatures can cause the olanzapine to be formed as, and/or converted to, olanzapine Form II. At the time of depressurizing, the temperature is generally not more than 60° C. and typically is within the range of 30° C. to 50° C.

In view of the above, the fluid composition prior to depressurization is preferably a supercritical fluid instead of merely a gas. In particular, the fluid composition typically comprises, or consists essentially of supercritical carbon dioxide containing olanzapine dissolved therein having a temperature in the range of 31-50° C., such as 35-45° C., and a pressure of 250-350 bars, i.e. about 300 bars.

To precipitate the olanzapine from the gas/supercritical fluid composition, the pressure of the fluid composition is sufficiently reduced. This pressure reduction lowers the solvation power of the gas/supercritical fluid composition thereby triggering precipitation of the olanzapine solute. The degree of the reduction in pressure and how it is achieved is not particularly limited. In general, larger reductions in pressure provide for a more efficient precipitation and higher yield of crystalline olanzapine; i.e., less olanzapine remains dissolved in the gas/supercritical fluid composition. Typically the pressure reduction is at least 50 bars, more typically at least 100 bars. In terms of relative pressure change, typically the reduction cuts the pressure at least in half, and more typically to one quarter or less of the starting pressure. In terms of values, the starting pressure is typically within the range of 200-400 bars and the ending pressure less than 50 bars, more typically 30 bars or less, and more commonly 20 bars or less.

The depressurization can be carried out by any suitable technique including increasing the volume of the fluid composition and/or transferring from a higher pressure zone to a lower pressure zone, etc. For example, a vessel containing the gas/supercritical fluid composition can be fitted with a piston and depressurization achieved by moving the piston to increase the volume of the vessel. Alternatively, the gas/supercritical fluid composition can be depressurized by venting the fluid composition to a lower pressure vessel/chamber or opening another vessel in fluid communication with the first vessel to increase the volume, etc. The depressurization is generally preferred to be rapid and dramatic, occurring in a single step, although this is not specifically required as slower and/or multi-step reduction could be used.

In view of the above, preferred techniques for depressurization fall under the category known as Rapid Expansion of Supercritical Solution or “RESS” techniques. The RESS technique is generally described by Tom and Debendetti in “Particle Formation with Supercritical Fluids—A Review”, J. Aerosol Sci., 22 (5), 555-584 (1991). In the pharmaceutical industry, the RESS technique has been used to form active pharmaceutical ingredients having a small particle size, e.g., see U.S. Pat. No. 5,795,594, and can form new polymorphic forms as in U.S. Pat. No. 6,406,718 where the novel Form 2 fluticasone propionate is described as being prepared by the RESS technique. In general the RESS technique involves adiabatic depressurization by jetting the supercritical fluid through an expansion nozzle into a vessel of lower pressure, typically sub-critical pressure. The expansion is so rapid that shock waves may be generated near the expansion nozzle. The size and dimension of the expansion nozzle is not particularly limited in the present invention and can be determined by routine skill. The RESS technique can also be generally applied to starting fluids that are gas compositions, especially to near supercritical gas compositions, and is not strictly used herein for supercritical fluid starting compositions.

Regardless of the depressurization technique, the temperature during precipitation is preferably not greater than 60° C., more preferably not greater than 50° C., in order to help insure the formation of Form I olanzapine.

Without wishing to be bound by any theory, it is suspected that the carbon dioxide molecule is the proper size to assist or facilitate the olanzapine to crystallize as Form I. But because carbon dioxide is a gas under normal conditions of temperature and pressure, it is not permanently trapped in or bound to the crystal lattice and thus does not form an olanzapine solvate. In any event and for whatever reason, olanzapine Form I can surprisingly be precipitated from a carbon dioxide-containing solvent.

The precipitated olanzapine is generally pure or substantially pure of other substances and is typically, though not necessarily, at least 97%, and usually at least 99% pure olanzapine. The precipitated olanzapine is also generally morphologically pure Form I olanzapine; e.g., at least 90% Form I olanzapine, preferably at least 95% Form I, more preferably at least 99% Form I, and most preferably essentially 100% Form I olanzapine, based on the total weight of crystalline olanzapine. Practically speaking, the Form I olanzapine produced by the present invention preferably shows no indication of Form II olanzapine, and more preferably no indication of any other Form of olanzapine, under x-ray powder diffraction analysis. The precipitated Form I olanzapine can be collected on a substrate/target, collected on a filter, collected from the bottom of the vessel, e.g. the bottom of the expansion chamber, etc.

After depressurization, the gas/supercritical fluid composition may be a gas or a supercritical fluid. Generally, the starting fluid composition is a supercritical fluid and the depressurized fluid composition is a gas. However, transitions from a supercritical fluid to another supercritical fluid as well as from a gas to another gas composition are contemplated as being used in the present invention. In terms of efficiency, a supercritical to gas transition is expected to yield the best results. The gas/supercritical fluid composition is ideally devoid of olanzapine after the depressurization, e.g., all of the olanzapine has been precipitated as crystalline Form I. Nonetheless, some olanzapine may remain in the gas/supercritical fluid composition after depressurization. If significant amounts of olanzapine remain, the fluid composition can be subject to additional depressurization step(s) and/or recycled. In any event, the depressurized, post-precipitation gas/supercritical fluid composition can generally be recycled and used again in the process. Typically such recycling can be achieved with relative ease in comparison to a liquid organic solvent recycle operation.

The starting gas/supercritical fluid composition containing carbon dioxide and olanzapine can be formed by any suitable technique. Typically the gas/supercritical fluid composition comprising or consisting of carbon dioxide is passed over solid olanzapine to extract the olanzapine into the fluid composition. Indeed, the use of supercritical fluids to carry out extraction is well known in the decaffeination of coffee as shown in U.S. Pat. No. 3,879,569. Higher pressures and temperatures provide for better extraction, e.g. faster uptake/higher olanzapine concentrations, and thus supercritical fluid compositions are generally preferred over sub-critical gas compositions when forming the starting fluid composition by extraction. Alternatively, the olanzapine can be dissolved in a liquid such as liquid carbon dioxide and then the composition converted such as by heating to form a gas/supercritical fluid composition. The starting olanzapine can be any form or purity, including technical grade olanzapine, Form II olanzapine, Form I olanzapine, or other hydrated or solvated forms of olanzapine.

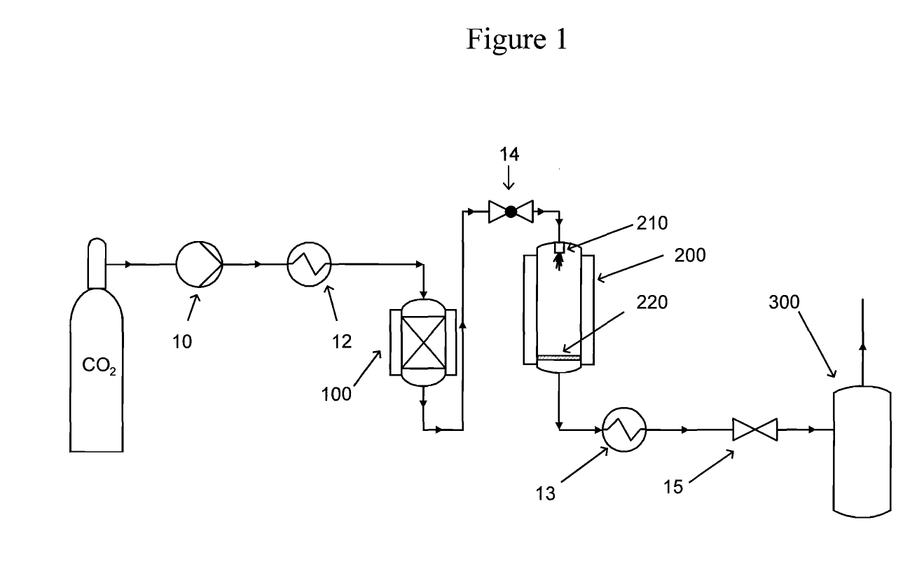

A preferred embodiment of the invention incorporating a RESS technique will be further described with respect to FIG. 1. FIG. 1 shows a schematic for carrying out a process of the invention using two pressure vessels. The first pressure vessel is the extractor 100. Carbon dioxide and optionally additional solvents, not shown, are pumped via pump 10 into the extractor 100 and maintained at a constant pressure, typically a supercritical pressure from 100 to 400 bars and preferably about 300 bars. The carbon dioxide and optional co-solvent are heated by heater 12 before entering the extractor 100. The pre-extractor heater and/or the heating jacket around the extractor maintain the temperature of the gas/supercritical fluid composition at the desired range, typically 30 to 50° C., e.g. about 40° C. Olanzapine powder is provided in the extractor between two filter plates, not shown, in an admixture with glass wool or similar material in order to maintain a loose powder structure. The olanzapine powder can be any form or purity of olanzapine such as technical grade olanzapine, etc. The carbon dioxide-containing gas/supercritical fluid composition passes through the filter plates and extracts olanzapine into the fluid composition.

The fluid composition then enters the second pressure vessel, a heated autoclave 200 that serves as an expansion chamber. The extractor 100 and autoclave 200 are connected via valve 14. The fluid composition is jetted into the autoclave through the expansion nozzle 210. The expansion nozzle has a diameter of about 200 microns. The autoclave has a temperature between 30 and 50° C. and preferably is about 40° C. For convenience, the extraction temperature and the depressurization temperature are approximately the same, e.g. within 4 degrees, more preferably within 2 degrees, of each other. However, a lower temperature can be used in the autoclave than in the extractor given that the solubility of olanzapine is intended to be reduced in the autoclave and that lower temperatures favor Form I olanzapine. The autoclave is kept at a lower pressure than the extractor, and is typically a value of 50 bars or less, more typically 20 bars or less. The sudden pressure drop causes a rapid and steep reduction of the solvent power of the carbon dioxide resulting in precipitation of the dissolved olanzapine as Form I. A filter 220 near the bottom of the autoclave collects the precipitated olanzapine. The low pressure fluid composition, now consisting primarily of carbon dioxide and the optional co-solvent is heated to about 100° C. upon leaving the autoclave via heater 13 to prevent the formation of dry ice and then expanded to atmospheric pressure in the flash vessel 300, located downstream of the back pressure valve 15. The carbon dioxide can be vented to the atmosphere or recycled to the carbon dioxide source. Alternatively, the low pressure carbon dioxide optionally with olanzapine can be recycled to the extractor via a high pressure pump to regain the starting pressure of the gas/supercritical fluid composition.

The precipitated olanzapine is Form I and typically is at least 99% pure Form I olanzapine and is typically substantially free from hydrates or solvates of olanzapine.

The invention will be further described by the following non-limiting example.

### EXAMPLE 1

Using an equipment scheme similar to that depicted in FIG. 1, a 500 ml extractor is filled to about half its volume with powdered olanzapine containing glass wool. The temperature of the pre-heater, the extractor, and autoclave are set to 40° C. and the pressure is built up with carbon dioxide (13.5 kg/hour) and maintained constant at 300 bars in the extractor and 20 bars in the autoclave. The ball valve before the autoclave is opened allowing the supercritical carbon dioxide containing olanzapine picked-up in the extractor to pass through the expansion nozzle, having a 200 micron diameter, and expand/decompress in the 6 liter autoclave. Olanzapine precipitates. The depressurized carbon dioxide fluid is allowed to leave the autoclave through the filter to the flash vessel via the post heater. The process is stopped by turning off the carbon dioxide supply and closing the ball valve before the autoclave. The heated autoclave is slowly depressurized and the product collected on the filter recovered. In about 30 minutes, about 0.72 grams of olanzapine Form I is formed.

Each of the patents, articles, and publications mentioned above is incorporated herein by reference in its entirety. The invention having been thus described, it will be obvious to the worker skilled in the art that the same may be varied in many ways without departing from the spirit of the invention and all such modifications are included within the scope of the present invention as set forth in the following claims.

We claim:

1. A process for making crystalline olanzapine Form I, which comprises reducing the pressure of a gas/supercritical fluid composition comprising carbon dioxide and olanzapine to precipitate crystalline olanzapine form I from said composition.

2. The process according to claim 1, wherein said composition is a supercritical fluid.

3. The process according to claim 1, wherein said composition comprises olanzapine dissolved in supercritical carbon dioxide.

4. The process according to claim 1, wherein said composition has a pressure of at least 60 bars.

5. The process according to claim 4, wherein said composition has a pressure of at least 100 bars.

6. The process according to claim 5, wherein said composition has a pressure of at least 200 bars.

7. The process according to claim 6, wherein said composition has a pressure of at least 300 bars.

8. The process according to claim 1, wherein said reduction in pressure step reduces the pressure of said composition by at least 50 bars.

9. The process according to claim 8, wherein said reduction in pressure step reduces the pressure of said composition by at least 100 bars.

10. The process according to claim 1, wherein said reduction in pressure step reduces the pressure of said composition to one fourth or less of the starting pressure.

11. The process according to claim 1, wherein said composition has a temperature within the range of about 30° C. to about 50° C.

12. The process according to claim 11, wherein said composition has a pressure in the range of 200 to 400 bars.

13. The process according to claim 12, wherein said reduction in pressure step reduces the pressure of said composition to 30 bars or less.

14. The process according to claim 1, wherein said reduction in pressure step is carried out by expanding the volume of said composition.

15. The process according to claim 1, wherein said reduction in pressure step is carried out by injecting said composition into a lower pressure zone.

16. The process according to claim 1, wherein said composition is formed by passing gaseous and/or supercritical carbon dioxide over solid olanzapine.

17. The process according to claim 1, wherein said precipitated crystalline olanzapine Form I has no detectable amount of Form II olanzapine under x-ray powder diffraction analysis.

18. The process according to claim 1, wherein said precipitated crystalline olanzapine Form I is at least 99% pure.

19. A gas/supercritical fluid composition comprising carbon dioxide and olanzapine and having a pressure of at least 100 bars and a temperature in the range of 31-50° C.

20. The composition according to claim 19, wherein said composition consists essentially of carbon dioxide and olanzapine and has a pressure within the range of 200-400 bars.
